(19) United States
(12) Patent Application Publication
(10) Pub. No.: US 2010/0286701 A1
(43) Pub. Date: Nov. 11, 2010

(54) DISTRACTION TOOL FOR DISTRACTING AN INTERSPINOUS SPACE

(75) Inventors: Christopher U. Phan, San Leandro, CA (US); Tanmay Mishra, Mountain View, CA (US)

Correspondence Address: MEDTRONIC;Attn: Noreen Johnson - IP Legal Department, 2600 Sofamor Danek Drive, MEMPHIS, TN, 38132, US

(73) Assignee: Kyphon SARL, Neuchatel (CH)

(21) Appl. No.: 12/463,134

(22) Filed: May 8, 2009

(51) Int. Cl. A61B 17/58

(52) U.S. Cl. 606/90

(57) ABSTRACT

Distraction tools for distracting an interspinous space formed between spinous processes. The distraction tool may include first and second handles connected together at a pivot between first and second ends. First and second prongs may extend outward from the handles. The handles may move about the pivot between a first orientation with the first and second prongs in proximity to each other and a second orientation with the first and second prongs separated in a first direction. A third member may be connected to the first and second handles and include a third outwardly-extending prong. The third member may be positioned together with the first and second prongs to form a unified prong when the first and second handles are in the first orientation. The unified prong is sized and shaped for insertion into the interspinous space. The third prong may separate from the first and second prongs in a second direction when the first and second handles are in the second orientation. The prongs distract the interspinous space when the handles are in the second orientation.

### BACKGROUND

Spinal implants are often used in the surgical treatment of spinal disorders such as degenerative disc disease, disc herniations, curvature abnormalities, and trauma. Further, as the present society ages it is anticipated that there will be an increase in adverse spinal conditions which are characteristic of older people. By way of example only, with aging comes increases in spinal stenosis (including, but not limited to, central canal and lateral stenosis), the thickening of the bones which make up the spinal column and facet arthropathy, and the narrowing of the spinal canal or neural foramen.

The implants may be inserted within an interspinous space formed between spinous processes. The implant affects the movement of the vertebral members and may control an extent of spinal movement during flexion, extension, and lateral bending. The implants may also control the relative positioning of the vertebral members.

It is necessary to distract the interspinous space prior to insertion the implant. This facilitates insertion and placement of the implant within the interspinous space. The distraction should provide for accurately placing the implant within the interspinous space, without overdistraction.

Current distraction tools often do not adequately distract the interspinous space, or are difficult for the surgeon to use. This makes placement of the implant difficult, and may cause damage to the vertebral members or the surrounding tissues. Further, current tools provide for a limited amount of distraction, such as in a single direction. This limited distraction may make the insertion process more difficult.

### SUMMARY

The present application is direction to distraction tools for distracting an interspinous space formed between spinous processes. The distraction tool may include first and second handles that each includes a first end and a second end. The handles may be pivotally connected together at a pivot between the first and second ends. First and second prongs may extend outward from the handles, and the prongs may include a tapered shape. The handles may move about the pivot between a first orientation with the first and second prongs in proximity to each other and a second orientation with the first and second prongs separated in a first direction. A third member may be connected to the first and second handles and includes a first connection with the first handle and a second connection with the second handle. The third member may include a third outwardly-extending prong. The third member may be positioned along the handles with the third prong together with the first and second prongs forming a unified prong when the first and second handles are in the first orientation. The unified prong is sized and shaped for insertion into the interspinous space. The third prong may separate from the first and second prongs in a second direction when the first and second handles are in the second orientation. The separation of the prongs in the first and second directions causes the distraction of the interspinous space.

The various aspects of the various embodiments may be used alone or in any combination, as is desired.

### BRIEF DESCRIPTION OF THE DRAWINGS

FIG. 1 is a perspective view of a distraction tool according to one embodiment.

FIG. 2 is a partial perspective view of distraction tool in a first closed orientation according to one embodiment.

FIG. 3 is a partial perspective view of distraction tool in a second open orientation according to one embodiment.

FIG. 4A is a schematic end view of the prongs in a first closed orientation according to one embodiment.

FIG. 4B is a schematic end view of the prongs in a second open orientation according to one embodiment.

FIG. 5 is a schematic view of the prongs in a second open orientation positioned within an interspinous space according to one embodiment.

FIG. 6 is a schematic view of a distraction tool in a first closed orientation according to one embodiment.

FIG. 7 is a schematic view of a distraction tool in a second open orientation according to one embodiment.

### DETAILED DESCRIPTION

The present application is directed to a distraction tool for distracting an interspinous space. The tool, generally illustrated as element 10, includes prongs 24, 32 that extend outward from a main body. The prongs 24, 32 are positioned together and form a unified prong when the tool 10 is in a first orientation. The unified prong facilitates insertion into the interspinous space. The tool 10 is movable to a second orientation with the prongs 24, 32 spaced apart to distract the interspinous space. Prongs 24 move apart in a first direction, and the prong 32 moves apart from prongs 24 in a second direction. The movement in the two directions provides for distracting the interspinous space.

The tool 10 generally includes a pair of handles 20 that are connected together at a pivot 23. Prongs 24 are positioned at the second ends 22 of the handles 20. A third member 30 is movably attached to the handles 20 and includes prong 32. The prongs 24 move apart due to the pivoting nature of the handles 20. The third member 30 moves apart by various structural configurations, including but not limited to a camming action with the handles 20, and a linkage configuration with the handles 20.

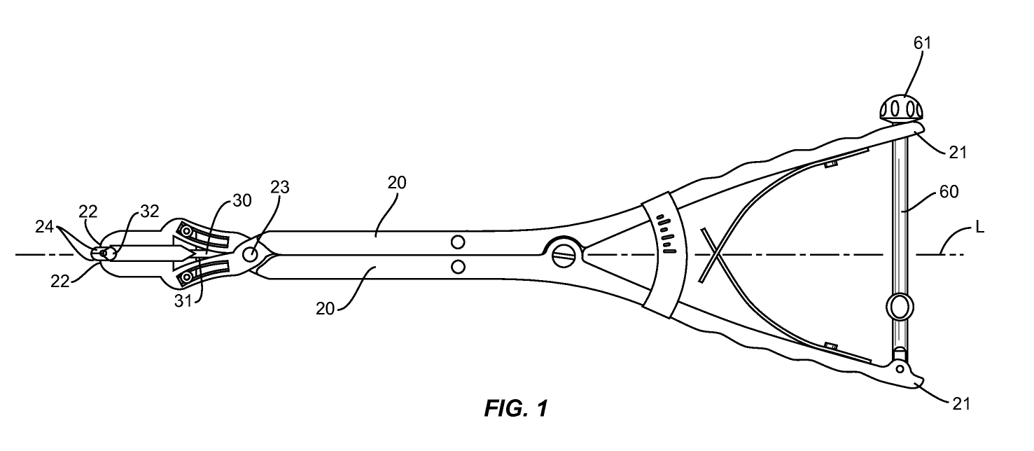

FIG. 1 illustrates the tool 10 with a pair of handles 20 connected together at the pivot 23. Each handle 20 includes a first end 21 and a second end 22. The pivot 23 connects the handles 20 at a point between the ends 21, 22. As illustrated in FIG. 1, the pivot 23 may be positioned in closer proximity to the second ends 22 than the first ends 21.

The prongs 24 are positioned at the second ends 22 of the handles 20. The prongs 24 may extend perpendicularly outward from a surface 53 of the handles 20 to allow the prongs 24 to be inserted into the interspinous space with the handles 20 remaining outside of the space. The prongs 24 may further include a tapered shape that reduces in cross-sectional size towards a tip 25. This tapered shape again facilitates insertion of the prongs 24 into the interspinous space.

The prongs 24 may also be shaped to form a unified single prong when the handles 20 are in the first orientation. The prongs 24 may include a curved exterior surface 50 positioned away from the opposing prong 24. The prongs 24 may also include a flat contact surface 51 positioned towards the opposing prong 24, and a second contact surface that faces towards the third member 30. The contact surface 51 of each prong 24 may be aligned perpendicularly to the second contact surface.

Each handle 20 may also include a slot 26 to receive the third member 30. The slots 26 include an inner surface 27 and an outer surface 28. The surfaces 27, 28 are parallel, and extend along a length of each handle 20. One or more shelves 29 may extend into the slots 26 from one or both of the surfaces 27, 28. The shelves 29 are positioned in the slots 26 away from the surface of the handles 20. The shelves 29 reduce a width of the slots 26.

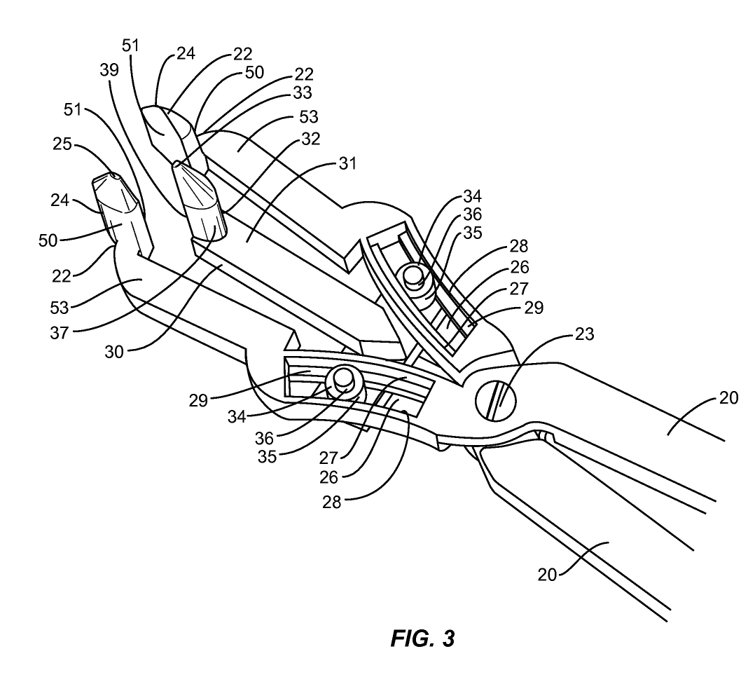

The slots 26 may be positioned at various locations along the length of the handles 20, including between the pivot 23 an the second ends 22. Further, the slots 26 may include various shapes, including curved and straight. FIGS. 1-3 include the slots 26 with a curved shape with the end closest to the pivot 23 being in closer proximity to the longitudinal axis L and the opposite end being farther from the longitudinal axis L.

The third member 30 is connected to the handles 20 and moves in the second direction away from the prongs 24 when the handles 20 move towards the second orientation. FIGS. 1-3 include the third member with a body 31 positioned between the handles 20. The prong 32 extends outward from body 31 and may include a tapered shape spaced away from the body 31 that terminates at a tip 33. The prong 32 may also include a curved outer surface 38 that faces towards the pivot 23 and a flat contact surface 39 on an opposite side.

The third member 30 may also include follower members 34 that engage with the slots 26 to move the third member 30. The follower members 34 may include posts 36 and roller bearings 35. The posts 36 extend outward from the body 31 and are sized to fit within the slots 26. The roller bearings 35 are rotatably positioned on the posts 36. The roller bearings 35 contact against the inner surfaces 27 of the slots 26 as the tool 10 moves between the orientations. The roller bearings 35 may also be sized to contact against the outer surfaces 28 of the slots 26. The roller bearings 35 may also be positioned on an upper surface of the shelves 29. The follower members 34 may include various other configurations, including but not limited to bearings, metallic balls, and posts.

The prongs 24, 32 are configured to extend into and distract the interspinous space. The length of the prongs 24, 32 may vary. As illustrated in FIGS. 1-3, the length is less than a distance between the pivot 23 and second ends 22 of the handles 20. Further, each of the prongs 24, 32 may include the same or different shapes and heights.

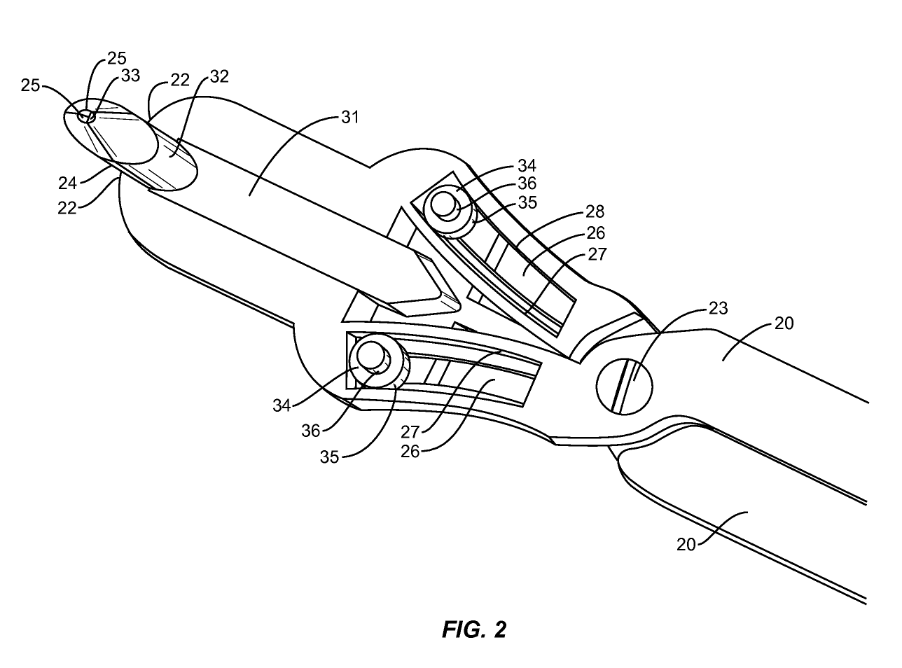

In use, the tool 10 is placed in the first closed orientation as illustrated in FIG. 2. This occurs by moving the first ends 21 of the handles 20 towards each other. This may be accomplished by a surgeon physically grasping the handles 20 and moving them together, or by rotating a threaded rod 60 connected to the first ends 21 of the handles 20. A knob 61 may be positioned at the end of the rod 60 to facilitate rotation.

The first orientation places the prongs 24, 32 together as illustrated in FIG. 2. This orientation forms a unified prong from the three separate prongs 24, 32. The unified prong is shaped and size for insertion into the interspinous space. The prongs 24, 32 may contact each other in this orientation, or may be spaced apart. In one embodiment, the flat contact surfaces 51 of the prongs 24 abut together and the flat contact surface 39 of the prong 32 abuts against the second contact surfaces of the prongs 24. The shapes of the prongs 24, 32 may also correspond with the curved outer surfaces 50, 37 and tapered sections matching together to reduce an overall size of the unified prong.

The tool 10 is manipulated by the surgeon while in the first closed orientation and the unified prong is inserted into the interspinous space. The unified prong extends outward with the main body remaining out of the interspinous space. The tapered shape of the unified prong may allow for easier penetration of the interspinous ligament without the need for a pilot hole before its insertion.

After insertion, the tool 10 is moved to the second open orientation to distract the interspinous space. This includes moving the first ends 21 of the handles 20 apart. Again, this force may be applied by the surgeon physically grasping the handles 20 and moving them apart, or by rotating a threaded rod 60 connected to the first ends 21 of the handles 20. The rod 60 may include a gauge that can be monitored to determine the spacing between the prongs 24, 32 while moving towards the second orientation. The spacing of the prongs 24, 32 in the second orientation may vary depending upon the size of the interspinous space. For some patients, this may include the prongs 24, 32 being positioned first distances apart, and for other patients it may include larger or smaller spacings.

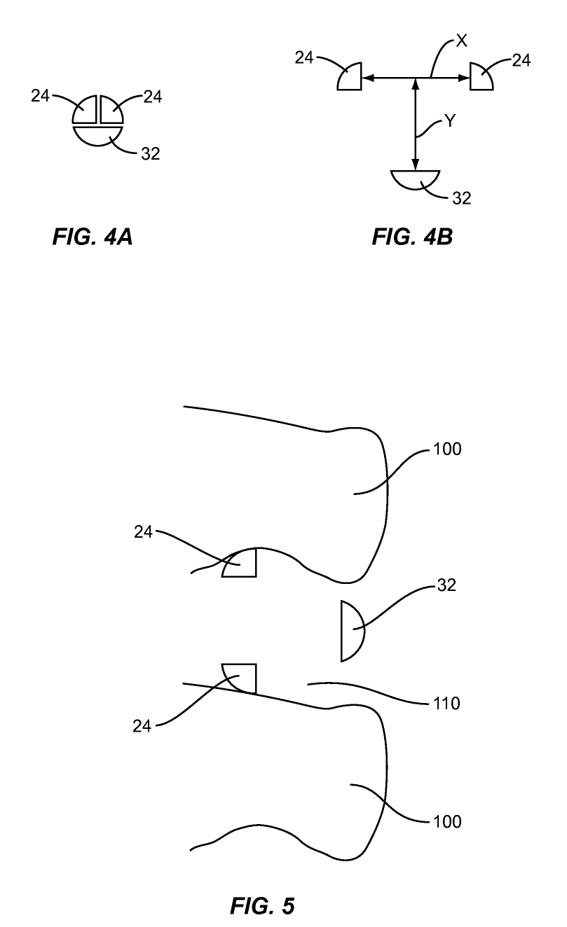

Movement of the tool to the second orientation causes distraction in two directions as illustrated in FIGS. 4A and 4B. FIG. 4A illustrates a schematic end view of the positioning of the prongs 24, 32 with the tool 10 in the first closed orientation. The prongs 24, 32 are positioned together to form the unified prong for insertion into the interspinous space. FIG. 4B illustrates the position of the prongs 24, 32 with the tool 10 in the second open orientation. Prongs 24 positioned on the handles 20 move apart in a first direction x. Prong 32 moves apart from the prongs 24 in direction y. The movement in the y direction is caused by the third member 30 moving towards the pivot 23. The direction y may be perpendicular to direction x. As illustrated in FIG. 4B, prong 32 is positioned between the prongs 24 in the direction x when the tool 10 is in the second open orientation. In one specific embodiment, the longitudinal axis L extends through the unified prong and the pivot 23 and the prong 32 moves along the longitudinal axis L during the movement between the first and second orientations. In one specific embodiment, prong 32 is positioned midway between the prongs 24 in the second orientation.

FIG. 5 schematically illustrates the prongs 24, 32 in an interspinous space while the tool 10 is in the second open orientation. The interspinous space 110 is formed between opposing spinous processes 100. The tool 10 is manipulated and placed into the interspinous space 110 such that the prongs 24 can distract the space 110 in an inferior-superior direction. Further, the prong 32 moves away from the prongs 24 to further distract the space 110 in an anterior-posterior direction. This distracted space is than able to receive an implant.

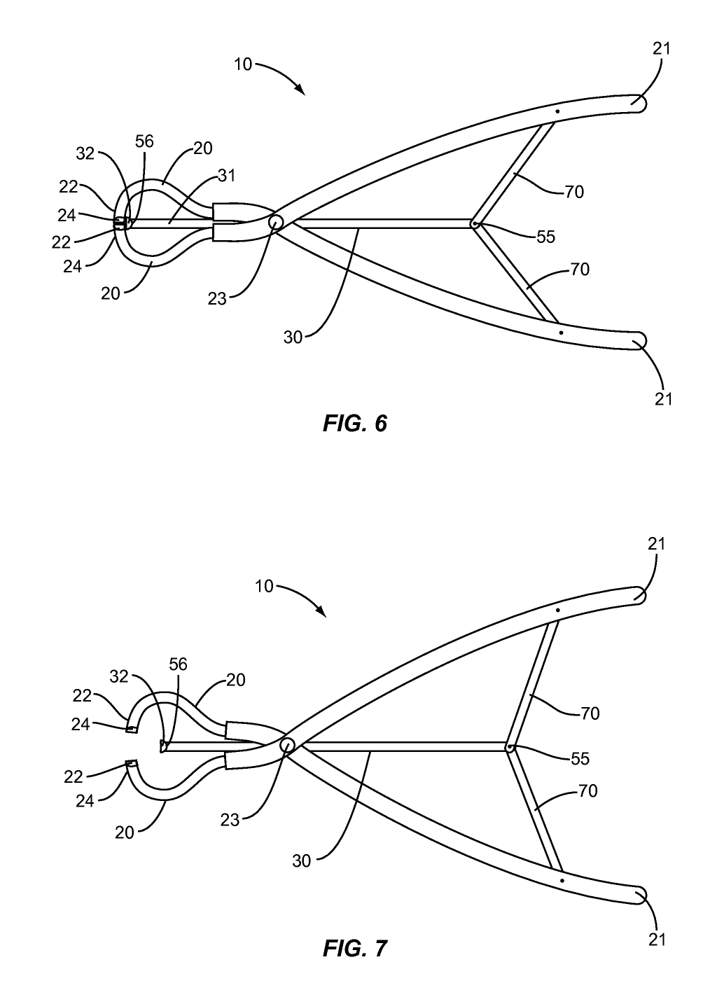

The third member 30 may also include a body 31 with an elongated shape that extends between first and second ends 55, 56 as illustrated in FIGS. 6 and 7. The body 31 extends along longitudinal axis L and may include a slot sized to receive the pivot 23. The slot includes an elongated shape larger than the pivot 23 to provide for movement of the body 31 when the tool 10 moves between the first and second orientations. Alternatively, the body 31 may be positioned beyond an end of the pivot 23 (i.e., the pivot 23 does not pass through the body 31). Links 70 extend from the handles 20 in proximity to the first ends 21 and connect with the body 31. The links 70 are positioned on an opposite side of the pivot from the prong 32.

The prongs 24, 32 in the second orientation form an opening in the interspinous space sized to receive an implant. The distracted opening may be the size of the implant. In one embodiment, the tool 10 is configured to accommodate an X-STOP implant available from Medtronic Spinal and Biologics of Sunnyvale, California. Examples of an X-STOP implant are disclosed in U.S. Pat. No. 6,695,842 herein incorporated by reference.

FIG. 6 illustrates the tool 10 in the first closed orientation. The prongs 24 are positioned together with the prong 32 on the third member 30 to form a unified prong. FIG. 7 illustrates the tool in the second open orientation with the ends 21 of the handles 20 being forced apart. This causes the handles 20 to move about the pivot 23 and separate the prongs 24 in the first direction. The force also causes the third member 30 to move with the prong 32 moving away in the second direction. The links 70 attached to the body 31 apply a force as the handles 20 are opened to move the third member 30 in the second direction.

A distraction tool is disclosed in U.S. Pat. No. 7,189,234 herein incorporated by reference in its entirety.

The distraction tool 10 may be used for distracting interspinous spaces at various levels along the spine. Further, the distraction tool 10 may access the interspinous spaces using various approaches (e.g., postero-lateral, lateral).

Leaf springs may be positioned between the handles 20 towards the first ends 21. The leaf springs apply a biasing force to bias the handles 20 towards the first orientation.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A distraction tool to distract an interspinous space comprising:
first and second handles each including a first end and a second end, the handles being pivotally connected together at a pivot between the first and second ends;
first and second prongs that extend perpendicularly outward from the second ends of each of the first and second handles;
the first and second handles movable about the pivot between a first orientation with the first and second prongs in proximity to each other and a second orientation with the first and second prongs separated in a first direction;
a third member movably connected to the first and second handles and including a first connection with the first handle and a second connection with the second handle;
a third prong that extends perpendicularly outward from the third member;
the third prong positioned together with the first and second prongs to form a unified prong when the first and second handles are in the first orientation, and the third prong separated from the first and second prongs in a second direction when the first and second handles are in the second orientation.

2. The distraction tool of claim 1, wherein the third prong is positioned midway between the first and second prongs when the first and second handles are in the second orientation.

3. The distraction tool of claim 1, wherein the first, second, and third prongs contact against each other to form the unified prong when the first and second handles are in the first orientation.

4. The distraction tool of claim 1, wherein each of the first and second handles includes a slot positioned between the second end and the pivot, and the first connection of the third member includes a first follower member positioned within the slot in the first handle and the second connection includes a second follower member positioned within the slot in the second handle.

5. The distraction tool of claim 1, wherein the third member includes an elongated shape with a first end and a second end positioned on opposite sides of the pivot, the third member being connected to the first and second handles with links.

6. The distraction tool of claim 1, wherein the first ends of the first and second handles are positioned in closer proximity with the handles in the first orientation than in the second orientation.

7. The distraction tool of claim 1, wherein the third member remains positioned between the second ends of the handles and the pivot when the first and second handles are in both the first and second orientations.

8. The distraction tool of claim 1, wherein the first direction is perpendicular to the second direction.

9. A distraction tool to distract an interspinous space comprising:
a first distracting prong extending outward from an end of a first handle;
a second distracting prong extending outward from an end of a second handle;
a pivot that connects the first and second handles together with the handles being movable about the pivot between closed and open orientations;
a third member connected to each of the first and second handles, the third member movable along a longitudinal axis between a first position when the handles are in the closed orientation and a second position when the handles are in the open orientation, the first position being in closer proximity to the first and second distracting prongs than the second position;
a third distracting prong extending outward from the third member;
the prongs being in contact to form a single member sized to be introduced into the interspinous space when the handles are in the closed orientation, the prongs being spaced apart in the open orientation with the first and second prongs positioned apart in a first direction and the third prong spaced apart from each of the first and second prongs in a second direction;
the longitudinal axis extending through the pivot and the single member when the handles are in the closed orientation.

10. The distraction tool of claim 9, wherein each of the prongs includes a tapered shape and the single member formed by the three prongs includes an overall tapered shape.

11. The distraction tool of claim 9, wherein each of the first and second handles includes slots and the third member includes cam members shaped to fit within and move along a length of the slots when the handles move between the closed and open orientations.

12. The distraction tool of claim 9, wherein the third member is positioned between the first and second prongs and the pivot when the handles are in both the closed and open orientations.

13. The distraction tool of claim 9, wherein the first prong extends perpendicularly outward from the first handle and the second prong extends perpendicularly outward from the second handle.

14. The distraction tool of claim 9, wherein the first direction is perpendicular to the second direction.

15. The distraction tool of claim 9, wherein the third prong is positioned halfway between the first and second prongs when the first and second prongs are separated in the first direction.

16. A method of distracting an interspinous space comprising:
positioning a main body in a closed orientation and inserting into an interspinous space first, second, and third prongs that each extend outward from the main body with the first, second, and third prongs being in contact and forming a single unified prong when the handles are in the closed orientation;
positioning the main body outside of the interspinous space while the first, second, and third prongs are in the interspinous space;
pivoting a pair of handles of the main body about a pivot and moving the first and second prongs apart in a first direction to distract the interspinous space and moving the third prong apart from the first and second prongs in a second direction to further distract the interspinous space.

17. The method of claim 16, further including distracting the interspinous space in a superior-inferior direction while moving the first and second prongs apart in the first direction, and distracting the interspinous space in an anterior-posterior direction while moving the third prong apart from the first and second prongs in the second direction.

18. The method of claim 16, wherein moving the third prong apart from the first and second prongs in the second direction includes sliding a third member of the main body along slots in each of the pair of handles.

19. The method of claim 18, further comprising moving the third prong apart from the first and second prongs and maintaining the third member between first and second prongs and the pivot.

20. The method of claim 16, further comprising contacting together flat surfaces of each of the first, second, and third prongs and forming the single unified prong when the handles are in the closed orientation.
